(19) United States
(12) Patent Application Publication
(10) Pub. No.: US 2022/0202887 A1
(43) Pub. Date: Jun. 30, 2022

(54) NEUROPROTECTIVE PHYLLANTHUS EMBLICA-CONTAINING COMPOSITIONS AND METHODS

(71) Applicant: Natreon, Inc., New Brunswick, NJ (US)

(72) Inventors: Sanyasi R. Kalidindi, Monroe, NJ (US); Deepaneeta Sarmah, Dwarka (IN); Pallab Bhattacharya, Muzaffarpur (IN); Kiran Kalia, Ahmedabad (IN)

(73) Assignee: Natreon, Inc., New Brunswick, NJ (US)

(21) Appl. No.: 17/558,900

(22) Filed: Dec. 22, 2021

(30) Foreign Application Priority Data
Dec. 24, 2020 (IN) 202041056217

(51) Int. Cl. A61K 36/185; A61P 25/28; A61P 9/10

(52) U.S. Cl. CPC A61K 36/185; A61P 9/10; A61P 25/28

(57) ABSTRACT

This invention is directed to methods of attenuating brain injury and providing neuroprotection to the brain from injury such as from stroke, by administering compositions containing extracts of Phyllanthus emblica; and related methods.

### CROSS-REFERENCE TO RELATED APPLICATIONS

This application claims priority to Indian Patent Application No. 202041056217, filed Dec. 24, 2020, which is incorporated by reference into this application in its entirety.

### FIELD OF THE INVENTION

This invention relates to methods of attenuating brain injury and providing neuroprotection to the brain from injury such as from stroke, with compositions containing extracts of Phyllanthus emblica.

### BACKGROUND

Stroke remains a foremost cause of death globally and is the primary cause of disability in the western world. Ischemic (occlusive clot-induced) stroke accounts for almost 85% of all cases of stroke. (Sarmah et al., “Mitochondrial dysfunction in stroke: implications of stem cell therapy” Translational Stroke Research 10:121-136 (2019); Saraf et al., “Intra-arterial stem cell therapy modulates neuronal calcineurin and confers neuroprotection after ischemic stroke” Int. J. Neurosci. 129(10):1039-1044 (2019b); Vats et al., “Intra-arterial Stem Cell Therapy Diminishes Inflammasome Activation After Ischemic Stroke: a Possible Role of Acid Sensing Ion Channel 1a” J. Mol Neurosci., doi: 10.1007/s12031-019-01460-3 (2019)).

Although ischemic stroke patients are often treated with thrombolytic agents, neuroprotection trials for stroke have been unsuccessful and, therefore, new pharmacological interventions are greatly needed. (Sarmah et al., “Getting closer to an effective intervention of ischemic stroke: the big promise of stem cell” Translational Stroke Research, 9:356-374 (2018); Sarmah et al., “Stroke Management: An Emerging Role of Nanotechnology” Micromachines (Basel) 8(9):262 (13 pages) (2017); Datta et al., “Cell Death Pathways in Ischemic Stroke and Targeted Pharmacotherapy” Transl. Stroke Res. doi: 10.1007/s12975-020-00806-z (2020); Kotian et al., “Evolving Evidence of Calreticulin as a Pharmacological Target in Neurological Disorders” ACS Chem Neurosci. 10(6):2629-2646 (2019)).

In the last decade, laboratory studies have suggested that components from plant origins are promising and can have an impact in the treatment of neurological disorders. (Pravalika et al., “Tigonelline therapy confers neuroprotection by reduced glutathione mediated myeloperoxidase expression in animal model of ischemic stroke” Life Sciences 216:49-58 (2019)). Phyllanthus emblica (P. emblica or Amla) has medicinal properties that are of paramount medicinal importance. (Thirunavukkarasu et al., “Protective effects of Phyllanthus emblica against myocardial ischemia-reperfusion injury: the role of PI3-kinase/glycogen synthase kinase 3β/β-catenin pathway” Journal of Physiology and Biochemistry 71:623-633 (2015)). P. emblica fruit is reported to contain polyphenolic compounds that act as antioxidants and may have a role in making the body's defense system robust. (Zhang et al., “Biological activities of phenolics from the fruits of Phyllanthus emblica L. (Euphorbiaceae)” Chemistry & Biodiversity 14:e1700404 (2017)).

In the past, P. emblica has shown benefits in treating renal disorders, inhibiting the proliferation of tumors, and in preventing diabetes. (Tasanarong et al., “Antioxidant effect of Phyllanthus emblica extract prevents contrast-induced acute kidney injury” BMC complementary and alternative medicine 14:1-11 (2014); Yahayo et al., “Suppression of human fibrosarcoma cell metastasis by Phyllanthus emblica extract in vitro” Asian Pacific Journal of Cancer Prevention 14:6863-6867 (2013); D'souza et al., “Anti-diabetic effects of the Indian indigenous fruit Emblica officinalis Gaertn: active constituents and modes of action” Food & Function 5:635-644 (2014)). It has also been shown to have immunomodulatory, anticancer, antioxidant and antiulcer activities (Zhao et al., “Anticancer properties of Phyllanthus emblica (Indian gooseberry)” Oxidative Medicine and Cellular Longevity 2015:950890 (2015); Varnosfaderani et al., “Efficacy and safety of Amla (Phyllanthus emblica L.) in non-erosive reflux disease: a double-blind, randomized, placebo-controlled clinical trial” Journal of Integrative Medicine, 16:126-131 (2018); Rajak et al., “Emblica officinalis causes myocardial adaptation and protects against oxidative stress in ischemic-reperfusion injury in rats” Phytotherapy Research: An International Journal Devoted to Pharmacological and Toxicological Evaluation of Natural Product Derivatives 18:54-60 (2004)). P. emblica has also been shown to act on the phosphoinositide 3-kinase/glycogen synthase kinase-3β (PI3K/GSK3β) signaling pathway in cardiac ischemia/reperfusion injury. (Thirunavukkarasu et al., 2015).

Mitochondrial dysfunction after an ischemic episode plays an important role in cerebral ischemic damage. Mitochondrial dysfunction includes a drastic change in activity of mitochondrial respiratory chain complexes, increased production of reactive oxygen species (ROS) and related cellular damage, mitochondrial swelling, and release of mitochondrial pro-apoptotic molecules among others. (Jordan et al., “Mitochondria: the headquarters in ischemia-induced neuronal death” Central Nervous System Agents in Medicinal Chemistry (Formerly Current Medicinal Chemistry-Central Nervous System Agents) 11:98-106 (2011)). A highly interconnected reticular mitochondrial network continuously undergoes cycles of fusion and fission as a part of performing normal physiological functions. (Perez-Pinzon et al., “Novel mitochondrial targets for neuroprotection” Journal of Cerebral Blood Flow & Metabolism 32:1362-1376 (2012)). Earlier studies have demonstrated that neuronal death following cerebral ischemia involves mitochondrial fission and preventing post-ischemic mitochondrial fission can lower cerebral ischemic damage. (Guo et al., “Drp1 stabilizes p53 on the mitochondria to trigger necrosis under oxidative stress conditions in vitro and in vivo” Biochemical Journal 461:137-146 (2014)). Protecting post-ischemic mitochondrial function can be an important strategy for post-ischemic neuroprotection.

### SUMMARY OF INVENTION

The present invention is directed to methods of providing neuroprotection and attenuating injury from stroke in the brain with Phyllanthus emblica-containing compositions. In an embodiment, the present invention is directed to a method of providing neuroprotection from stroke injury in the brain of a subject comprising the steps of (a) providing a composition comprising a Phyllanthus emblica extract, and (b) administering an effective amount of the composition to the subject to act on the subject's brain and provide neuroprotection from stroke injury in the brain.

In an embodiment, the present invention is directed to a method of attenuating brain injury from stroke in a subject comprising the steps of (a) providing a composition comprising a Phyllanthus emblica extract, and (b) administering an effective amount of the composition to the subject to act on the subject's brain and attenuate injury from stroke in the subject's brain.

In an embodiment, a method of this invention comprises administering a composition comprising a standardized aqueous extract of Phyllanthus emblica such as Capros®. In an embodiment, the P. emblica extract may be administered before, during, and/or after a stroke, including for instance less than 1 hour after interruption of blood flow to the brain, or for instance less than 1 hour after resumption of blood flow to the brain.

In an embodiment, a method of this invention comprises providing neuroprotection or attenuating injury from a cognition-related disease or disorder in the brain of a subject comprising the steps of (a) providing a composition comprising a Phyllanthus emblica extract, and (b) administering an effective amount of the composition to the subject to act on the subject's brain and provide neuroprotection and/or attenuate injury from cognition-related disease or disorder in the brain. In an embodiment, the present methods provide neuroprotection or attenuate brain injury in mild cognitive impairment, or dementia such as Huntington's disease, Alzheimer's disease, and/or vascular dementia.

### BRIEF DESCRIPTION OF THE DRAWINGS

FIG. 1 in an embodiment is a chart showing changes in cerebral blood flow during middle cerebral artery occlusion (MCAO) surgery, as measured by Laser Doppler Flowmetry.

FIG. 2 in an embodiment is a chart showing attenuation of brain injury and showing neuroprotection with administration of a P. emblica-containing composition of the present invention in view of statistically significant improvement in rotarod performance by rats 24 hours following cerebral ischemic stroke.

FIG. 3 in an embodiment is a chart showing attenuation of brain injury and showing neuroprotection with administration of a P. emblica-containing composition of the present invention in view of statistically significant improvement in grip strength of rats 24 hours following cerebral ischemic stroke.

FIG. 4 in an embodiment is a chart showing attenuation of brain injury and showing neuroprotection with administration of a P. emblica-containing composition of the present invention in view of statistically significant improvement in neurological deficit score of rats 24 hours following cerebral ischemic stroke.

FIG. 5A in an embodiment represents photomicrographs of coronal slices of cortical rat brain with TTC (Triphenyl tetrazolium chloride) staining, with reduced infarct size in rats treated with P. emblica-containing compositions before and after stroke.

FIG. 5B in an embodiment is a chart showing attenuation of brain injury and showing neuroprotection with administration of a P. emblica-containing composition of the present invention in view of statistically significant reduction in cerebral infarct size in rats 24 hours after cerebral ischemic stroke.

FIG. 6 in an embodiment is a chart showing attenuation of brain injury and showing neuroprotection with administration of a P. emblica-containing composition of the present invention in view of statistically significant reduction in GSH depletion in cortical rat brain 24 hours following cerebral ischemic stroke.

FIG. 7 in an embodiment is a chart showing attenuation of brain injury and showing neuroprotection with administration of a P. emblica-containing composition of the present invention in view of statistically significant inhibition of nitrite generation in cortical rat brain 24 hours following cerebral ischemic stroke.

FIG. 8 in an embodiment is a chart showing attenuation of brain injury and showing neuroprotection with administration of a P. emblica-containing composition of the present invention in view of statistically significant inhibition of lipid peroxidation in cortical rat brain 24 hours following cerebral ischemic stroke.

FIG. 9 in an embodiment is a representation of a Western blot showing the expression of TOMM20 protein, a marker for mitochondrial outer membrane.

FIG. 10 in an embodiment is a chart showing attenuation of brain injury and showing neuroprotection with administration of a P. emblica-containing composition of the present invention in view of statistically significant inhibition of mitochondrial complex I dysfunction in cortical rat brain 24 hours following cerebral ischemic stroke.

FIG. 11 in an embodiment is a chart showing effects of P. emblica-containing compositions on mitochondrial complex II activity in cortical region of rat brain 24 hours following cerebral ischemic stroke.

FIG. 12 in an embodiment is a chart showing attenuation of brain injury and showing neuroprotection with administration of a P. emblica-containing composition of the present invention in view of statistically significant inhibition of mitochondrial complex IV dysfunction in cortical rat brain 24 hours following cerebral ischemic stroke.

FIG. 13 in an embodiment is a chart representing the respiratory control ratio (RCR) between Sham, Stroke, Prophylaxis, and Treatment groups.

FIG. 14 in an embodiment is a graph showing representative High-Resolution Respirometry of mitochondrial preparations and relevant substrates.

FIG. 15 in an embodiment is a representation of Western blots showing the expression of various proteins.

FIG. 16 in an embodiment is a series of charts showing protein expression changes post-stroke in rats administered P. emblica-containing compositions.

### DETAILED DESCRIPTION OF THE INVENTION

The below definitions and discussion are intended to guide understanding but are not intended to be limiting with regard to other disclosures in this application. References to percentage (%) in compositions of the present invention refers to the % by weight of a given component to the total weight of the composition being discussed, also signified by “w/w”, unless stated otherwise. Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

A “composition” of the present invention comprises an extract from the fruit of Phyllanthus emblica (Emblica officinalis). A composition of the present invention may comprise, consist essentially of, or consist of, an extract of P. emblica fruit.

In the present invention, an “extract” is prepared from P. emblica fruit by disrupting the fruit from its natural state and treating the fruit with water or aqueous solution such as phosphate buffered saline (PBS) or other aqueous solution with for instance a salt, pH, and/or other chemical component(s) to form the aqueous extract. In the present invention, a “standardized aqueous extract” is an extract in which specific components have been identified and present in a minimum or maximum amount or a specific range, so as to render the extract consistent at least with regard to those components from one batch to the next. In an embodiment an extract, preferably a standardized aqueous extract, of P. emblica fruit is 60% (w/w) or greater low molecular weight hydrolyzable tannins, for instance 70% (w/w) or greater, or 80% (w/w) or greater; and/or about 5% (w/w) or less gallic acid, for instance 4% (w/w) or less, or 3% (w/w) or less, or 2% (w/w) or less gallic acid. In an embodiment, low molecular weight refers to a molecular weight of less than 1000 daltons.

In an embodiment, a standardized aqueous extract of this invention is prepared by extracting finely pulped P. emblica fruit with a dilute aqueous or alcoholic-water salt solution, for instance 0.1-5% (w/w) sodium chloride solution and/or 0.1-5% (w/w) sodium citrate/citric acid, or another salt, preferably at a temperature of about 70° C. (e.g. 65-75° C.) to form an extract-containing solution, filtering the solution, and drying to provide the extract as a powder. In an embodiment, 1% NaCl(w/w) is used. In an embodiment, one or more processes for preparing a P. emblica extract of the present invention is described in U.S. Pat. No. 6,124,268, and is incorporated by reference herein to describe said process(es).

In an embodiment, a standardized aqueous extract of P. emblica fruits according to this invention is Capros® (Natreon, New Brunswick, N.J.). Capros® is a preferred extract of P. emblica fruit of this invention, and is the P. emblica extract used in the below Example. Capros® is a super antioxidant, completely water soluble and stable, suitable for solid dosage forms such as powdered forms, for instance for hot and cold beverages. In an embodiment, Capros® has the appearance of a yellow free-flowing powder, with an astringent taste. The powder has a water-soluble extractive value of greater than or equal to 80% (w/w). In an embodiment, said value is greater than 90%, or greater than 95%. In an embodiment, Capros® powder includes greater than or equal to 60% (w/w) low molecular weight hydrolysable tannins, including for instance greater than 70% or greater than 75%; has a gallic acid content less than or equal to 4% (w/w), including for instance less than 2%, or less than 1%; and in an embodiment further has a water content of less than or equal to 6% (w/w), including less than 5% w/w, less than 4% w/w, less than 3% w/w, or less than 2% w/w; and has a sulfated ash content of less than or equal to 6% (w/w). In an embodiment such as the Capros® used in the below Example, Capros® has a low molecular weight hydrolysable tannin content of about 71%, gallic acid content of about 0.17%, about 16-17% mucic acid-2-O-gallate, about 4% Mucic acid-1,4-lactone-5-O-gallate, and about 16-17% galloyl glucose, with 90% or more particles passing through 40 mesh size, and 80% or more particles passing through 80 mesh size, bulk density of about 0.56 g/cc (within an acceptable range of 0.4-0.75 g/cc), moisture content about 4%, sulfated ash about 5%, and water-soluble extractive value about 88%. In an embodiment, Capros® includes fewer than 10 ppm heavy metals, for instance less than 2 ppm; 5000 CFU/g aerobic bacteria or less (including for instance less than 1000 CFU/g or less than 20 CFU/g); and no measurable Escherichica coli or Candida albicans in 1 g powder, or Salmonella species, Pseudomonas aeruginosa, and/or Staphylococcus aureus in 10 g powder.

In an embodiment, Capros® is prepared by washing and de-pulping the fresh P. emblica fruits, pressing and centrifuging the pulp to squeeze the juice out, mixing the juice with small percentages of sodium chloride to prevent oxidative decomposition, sodium benzoate or a natural preservative to prevent bacterial growth, and optionally 10-30% maltodextrin as a carrier and silicon dioxide as an anti-caking and anti-sticking agent. The mixture is then spray-dried into a powder and stored.

In an embodiment, a standardized aqueous extract of this invention is in powdered form and may be blended together with other substances in powdered form. In another embodiment, the aqueous standardized extract may be in liquid form, for instance as prepared or for instance as a powder dissolved into water or other liquid. A composition of the present invention may further comprise one or more excipients, additives, and/or other substances, including for instance microcrystalline cellulose, croscarmellose sodium, magnesium stearate, and/or silicon dioxide; and/or a suitable aqueous solution such as a buffer solution. A composition of the present invention may be formulated into nutraceutical or pharmaceutical dosage forms comprising for instance tablets, capsules, powders, liquids, chews, gummies, lozenges, pills, and so forth. In an embodiment, a composition of the present invention is the composition administered as in the Example below, and/or used to prepare the composition.

In an embodiment, a composition comprising an extract such as a standardized aqueous extract of this invention, preferably Capros®, is administered in an effective amount to a subject, including a daily dose of P. emblica extract for a human being of at least 1-10,000 mg, in an embodiment at least 50 mg, 75 mg, 100 mg, 200 mg, 300 mg, 400 mg, 500 mg, 1000 mg, 1500 mg, 2000 mg, 5000 mg, 8000 mg, 10,000 mg, and/or any range or amount within the range of 1-10,000 mg, including for instance 50 mg-8000 mg, 100 mg-2000 mg, 200 mg-800 mg, and other internal ranges. In an embodiment, a dose of P. emblica extract in a human being may be 1 mg/kg to 2 g/kg, including for instance 2-10 mg/kg, 50 mg/kg-200 mg/kg, or 100 mg/kg. In a non-human animal, a daily dose may be about the same as in a human, adjusted per kilogram of weight of the animal, for instance 100 mg P. emblica extract/kg subject as described in the Example below.

While an extract according to the present invention may be an aqueous or standardized aqueous extract, in an embodiment, an extract of the present invention may be prepared without water or aqueous solution, and/or without being standardized.

In the present invention, a “dietary supplement” refers to a composition comprising Phyllanthus emblica extract which is administered as an addition to a subject's diet, which is not a natural or conventional food. In an embodiment, a dietary supplement administered to a subject includes an effective amount of Phyllanthus emblica extract, such that the Phyllanthus emblica-containing composition enters the body and may be acted upon by the body, and reaches blood and/or tissues and/or cells of the subject's body (in particular the brain) to provide neuroprotection and/or attenuate brain injury from stroke in the subject's brain, and otherwise act for instance as discussed throughout this application. In an embodiment, a dietary supplement containing an effective amount of Phyllanthus emblica according to the present invention is administered orally. In an embodiment, a dietary supplement or other composition of this invention is administered daily. In an embodiment, the dietary supplement is administered daily for 1 day, 1-7 days, 1-14 days, 1-30 days, 30 days, 30-60 days, or for another period of time according to the present invention. In an embodiment, a dietary supplement according to this invention may be taken chronically, for instance for several months or a year or years. A dietary supplement may be formulated into various forms, such as a powdered form, and otherwise as discussed throughout this application.

In the present invention, “administering”, “administration”, and the like, refers to providing a composition of the present invention to a subject so that the Phyllanthus emblica extract is present in an amount effective to enter the subject's body and reach the subject's bloodstream and/or tissues and/or cells in the brain and act on the subject's brain (e.g. tissues and cells) to provide neuroprotection in the brain and/or attenuate brain injury, for instance as discussed throughout this application including in the Example (attenuate and/or protect from impairment of motor function, sensory function, and/or balance by stroke; attenuate and/or protect from decreases in GSH, increases in oxidative and/or nitrosative stress, mitochondrial dysfunction, from stroke). In an embodiment, the low molecular weight hydrolysable tannins are active components of the P. emblica extract that act in the subject's body to provide neuroprotection and attenuate injury from stroke. Administration may occur before, during, and/or after the occurrence of a stroke, for instance, at any time before the interruption of blood flow and/or reperfusion of brain tissue, for instance, 0-24 hours before, about 1 day before, and administered daily for instance for 1-7 days before, about 1 week before, 1-30 days before, about 30 days before, or more. Administration may also occur during interruption of cerebral blood flow and/or during the reperfusion period of the stroke, and/or may occur after the occurrence of a stroke, for instance, within 1 hour of the interruption of cerebral blood flow and/or within 1 hour of removal of the occlusion and beginning of reperfusion, within 0-2 hours after stroke, 0-3 hours, 0-4 hours, 0-5 hours, or within for instance 1 day of the stroke. Administration may be chronic, for instance, more than 2 months, 6 months, or a year or more. Administration may be by the subject or by another. Administration may be oral, for instance in the form of a dietary supplement in a solid dosage form such as a powder or mixed into a beverage or as a discrete dose unit such as a capsule, and/or administered via other routes in physiologically acceptable forms, such as rectally as a suppository, according to the present invention. In an embodiment, a composition of this invention, such as Capros®, will be taken orally either before or after a meal, or rectally in the form of a suppository. In an embodiment, administration according to this invention is as described in the below Example (dissolving the standardized P. emblica extract into normal saline and administering orally).

In the present invention, a “subject” is a human being, a rat, a horse, a dog, a cat, or other mammal or other animal having a brain in which injury from a stroke may occur.

“Co-administration” refers to administering a composition of the present invention with another substance, for instance, a drug that provides neuroprotection and/or a drug that treats stroke and/or brain injury from a stroke, such as for example clopidogrel and/or aspirin. In an embodiment, such co-administration may be at different times, so long as both extract and drug are available in the brain of the subject in an effective amount to provide neuroprotection and/or attenuate injury from stroke.

In the present invention, an “effective amount” of Phyllanthus emblica-containing composition refers to an amount of Phyllanthus emblica extract of this invention needed to be administered to a subject in order to reach a subject's bloodstream and/or bodily tissues and cells and to provide neuroprotection, attenuate brain injury in the subject's brain from stroke, and/or otherwise act for instance as discussed throughout this application. In an embodiment, an effective amount of Phyllanthus emblica extract is a daily dose as discussed above or throughout this application. In an embodiment, an effective amount of Phyllanthus emblica-containing composition according to this invention is about 100 mg/kg as discussed in the Example below. In an embodiment, an effective amount of Phyllanthus emblica-containing composition is 1-10,000 mg of P. emblica extract/day, for instance 50, 100, 200, 300, 400, 500, 600, 700, 800, 900, 1000, 1500, 2000 mg/day, including any amount or range within said amounts.

In the present invention, “stroke injury” or “injury from a stroke” and the like refers to injury in the brain of a subject caused by an interruption in blood flow to the brain, and later resumption of blood flow, for instance by a blockage as in ischemic stroke or transient ischemic attack. Injury from a stroke may cause damage to a subject's brain tissue and brain cells, for instance by forming an infarct in the brain and/or increasing infarct size, increasing oxidative and/or nitrosative stress, and/or causing mitochondrial dysfunction. Injury from a stroke may cause damage to a subject's brain and impair for instance sensory function, motor function, and/or balance. Injury from a stroke may be measured for instance by tests of balance (e.g. rotarod test), motor function (e.g. grip strength), neurological deficit (e.g. sensory function, motor function, balance), brain tissue damage (e.g. infarct size), oxidative and/or nitrosative stress (e.g. tests of GSH levels, nitrite levels, MDA levels), mitochondrial dysfunction, and/or by evaluating changes in protein levels from brain injury.

In the present invention, “neuroprotection”, “neuroprotective” and the like refers to protecting the brain, brain cells such as neurons and/or related brain cells and/or tissue, from injury from stroke with the administration of a P. emblica extract of this invention. Such neuroprotection may include for instance halting, avoiding, and/or reducing injury to a subject's brain from a stroke; including injury such as impairment of sensory function, motor function, and/or balance; formation and/or increased size and/or development of an infarct; increased oxidative and/or nitrosative stress; and/or mitochondrial dysfunction. In an embodiment, neuroprotection via administration of a P. emblica composition according to this invention is evidenced by comparison with stroke injury in those that were not administered a P. emblica composition of this invention or by comparison with pre-stroke characteristics of the subject or of a group. In an embodiment, and without being bound by theory, the present invention provides neuroprotection for instance by increasing the expression of neurotrophic factors such as SDF-1, BDNF, and VEGF; regulating neuronal growth and axonal regeneration; and upregulating components of the PI3K/Akt/GSK3b pathway.

In the present invention, “attenuate”, “attenuating” and the like refer to halting, avoiding and/or reducing injury from stroke in the brain of a subject with the administration of a P. emblica composition according to the present invention; including injury such as impairment of sensory function, motor function, and/or balance; formation and/or increased size and/or development of an infarct; increased oxidative and/or nitrosative stress; and/or mitochondrial dysfunction. In an embodiment, attenuation of stroke injury via administration of a P. emblica composition is evidenced by comparison with stroke injury in subjects that were not administered a P. emblica composition of this invention, or by comparison with pre-stroke characteristics of the subject. In an embodiment, and without being bound by theory, the present invention attenuates brain injury for instance by increasing the expression of neurotrophic factors such as SDF-1, BDNF, and VEGF; regulating neuronal growth and axonal regeneration; and upregulating components of the PI3K/Akt/GSK3b pathway.

Neuroprotection from and/or attenuation of brain injury from stroke according to this invention is evidenced for instance in the below Example. In an embodiment, the Treatment group in the below Example attenuates injury from stroke in the brain and provides neuroprotection to the brain by orally administering an effective amount of a standardized extract of P. emblica (Capros®) after ischemic stroke in the brain. In an embodiment, the Prophylactic group in the below Example attenuates stroke injury in the brain and provides neuroprotection to the brain by orally administering an effective amount of a standardized extract of P. emblica (Capros®) before ischemic stroke in the brain. In an embodiment, references throughout this application to attenuation and neuroprotection according to the present invention may include treatment such as in the Treatment group in the Example, and/or prophylactic treatment such as in the Prophylaxis group in the below Example, with a standardized extract of P. emblica (e.g. Capros®).

In an embodiment, neuroprotection from and/or attenuation of brain injury from stroke with administration of a P. emblica extract such as Capros® according to this invention includes reducing injury to a brain structure, function, activity, and/or other negative impact on the subject's brain from stroke by about 5% to 500% or more, including any range or number including or falling within this range, including for instance from 25% to 50%, 40% to 60%, 50% to 75%, 70% to 90%, 85% to 100%, 100% to 400%, and so forth, compared with pre-stroke brain normal for the subject or for an average group of subjects. In addition, in an embodiment, neuroprotection from brain injury from stroke includes (but is not limited to) an improvement in the subject's brain activity, function, and/or structure with P. emblica administration, for instance by about 1% to about 30% or more, preferably about 5% to about 20%. In an embodiment, this improvement may be measured pre-stroke as well as post-stroke.

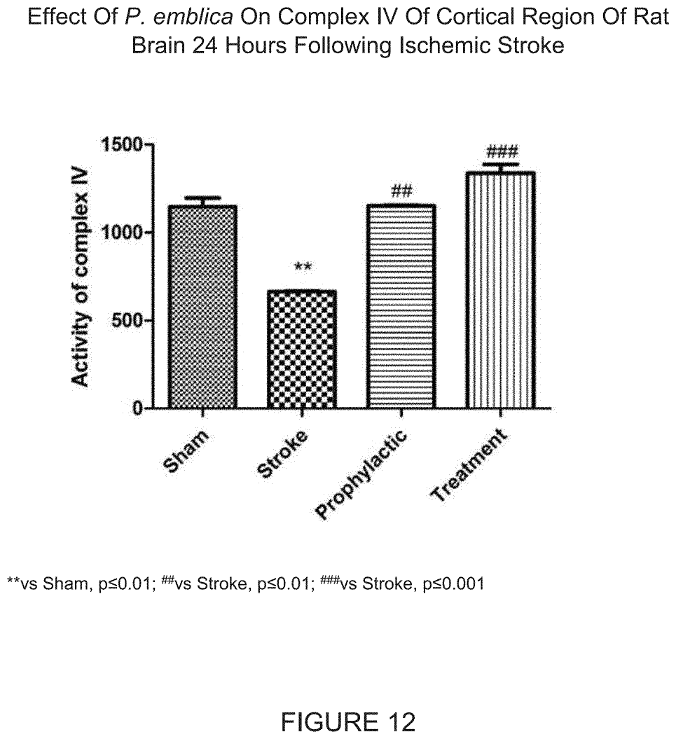

In an embodiment, neuroprotection from and/or attenuation of brain injury after a stroke includes halting and/or avoiding injury to the brain, for instance maintaining the structure, activity, and/or function present in the subject's brain before the stroke (pre-stroke). See for instance FIG. 12 and related discussion below, showing neuroprotection from and attenuation of brain injury after stroke. The activity of mitochondrial complex IV in the post-stroke Prophylactic and Treatment groups of animals administered P. emblica extract (Capros®) is about the same (approximately 0% loss in activity/injury from stroke, or 100% maintenance of activity) in Complex IV activity as the control Sham group, in contrast to the 60% drop in Complex IV activity manifested in the untreated Stroke group as injury in the brain from stroke. The Treatment group also indicates improvement in the subject's Complex IV activity over control, showing neuroprotection with Capros® administration in the Treatment group.

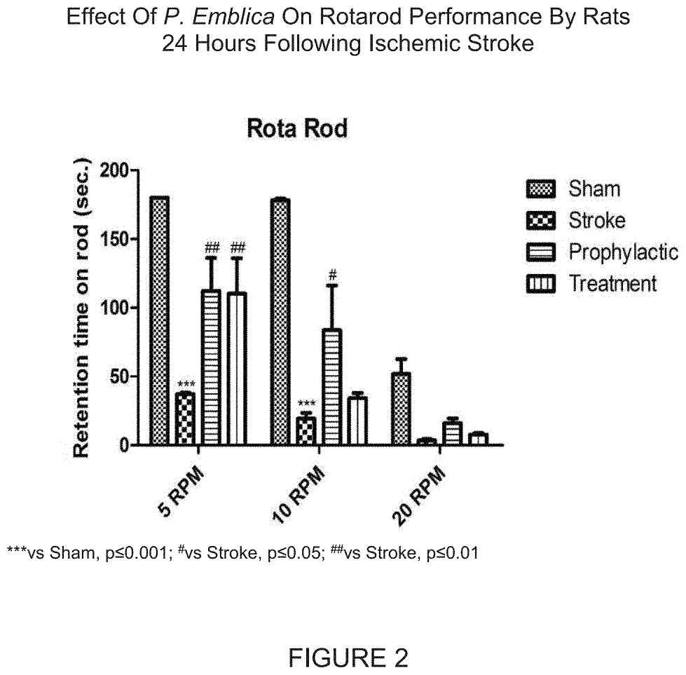

See also for instance FIG. 2 and related discussion below, showing neuroprotection from and attenuation of brain injury after stroke. The retention time on the rotarod at 5 RPM shows, columns left to right, about 180 seconds (control “Sham” group, with no stroke induction and no P. emblica administration), about 40 seconds (control “Stroke” group, with stroke induction and no P. emblica administration), and about 110 seconds (with stroke induction, Capros®-treated Prophylactic and Treatment groups). Neuroprotection from and attenuation of brain injury in FIG. 2 is evident from the 50% attenuation (reduction) of brain injury seen in Prophylactic and Treatment groups as compared with untreated control Stroke rats ((40−110)/(180−40)=−50%). Similarly, the 10 RPM data in FIG. 2 shows statistically significant reduction of brain injury in Prophylactically treated rats by about 34% as compared with injury in untreated control Stroke rats ((20−75)/(180−20))=−34%) (with 10 RPM retention time columns, left to right, showing about 180 seconds (Sham), 20 seconds (Stroke), and 75 seconds (P. emblica-treated Prophylactic)). Other calculations may be used to help describe neuroprotection and attenuation of brain injury post-stroke according to the other Figures and disclosures in this application.

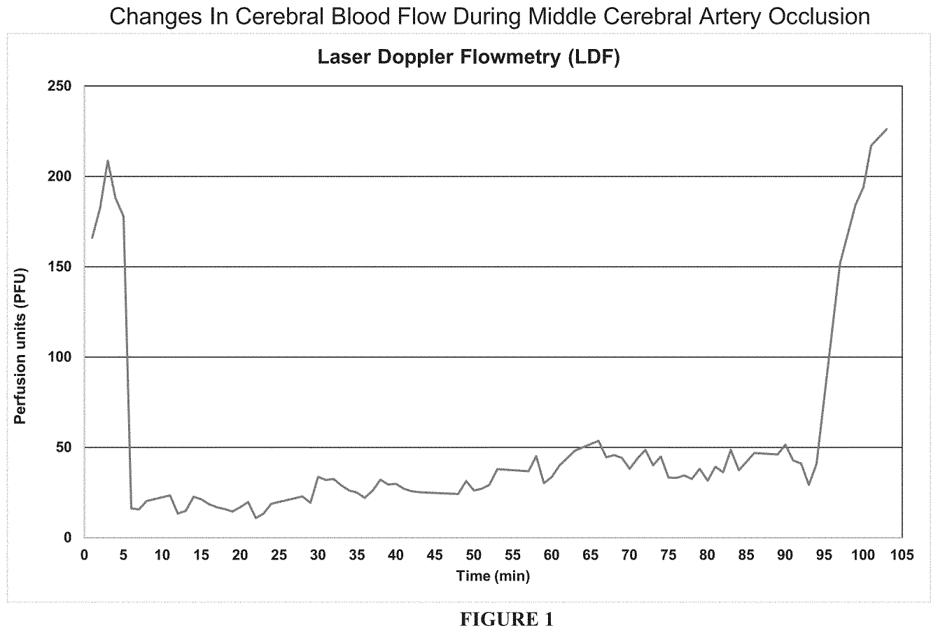

P. emblica-containing compositions of this invention such as Capros® may provide neuroprotection and/or attenuate injury to the brain for instance by increasing the expression of neurotrophic factors like SDF-1, BDNF and VEGF; regulating neuronal growth and axonal regeneration as demonstrated by the increased expression of GAP-43, and in an embodiment, facilitating neurogenesis; and upregulating the PI3K/Akt/GSK3β pathway, in addition to other forms of neuroprotection from and attenuation of stroke injury in the brain shown for instance in FIGS. 1-12. The neuroprotective effects of a P. emblica-containing composition of this invention are further confirmed by attenuation of injury and by improvement in motor-functional coordination, reduction in infarct size, improvement in oxidative stress outcomes, and other improvements in stroke outcomes as described throughout this application.

Without being bound by theory, neuroprotection and/or attenuation of injury in the brain with a composition comprising a P. emblica extract according to this invention appears to increase and/or fortify protections in brain cells and/or tissues against oxidative or nitrosative attack or other forms of injury, whether administered before stroke or after stroke. Without being bound by theory, in an embodiment, components of P. emblica extract of this invention cross the blood-brain barrier to provide neuroprotection to the brain or to attenuate injury in the brain. In an embodiment, a method of this invention may include attenuating injury and providing neuroprotection from brain injury similar to stroke such as multi-infarct dementia, as well as from other diseases and disorders in which subjects have or develop a cognition-related disease or disorder, such as mild cognitive impairment or dementia including dementia from Huntington's disease, Alzheimer's disease, or vascular dementia. In an embodiment, a method of this invention is a method of treating and/or preventing injury to the brain from a stroke and/or treating and/or preventing a cognition-related disease or disorder and/or one or more symptoms thereof, for instance by providing neuroprotection and/or attenuating injury in the brain of a subject in need thereof, for instance as discussed throughout this application.

In the present invention, reference to “significant” findings are to findings marked with a statistical “p” value less than or equal to 0.05 (p≤0.05). References to p≤0.01 and p≤0.001 are less than 0.05, and thus also statistically significant findings. The lack of an indicator of statistical significance is not intended as determinative unless expressly noted or indicated so.

The present invention may be further understood in connection with the following Example and embodiments. The following non-limiting Example and embodiments described throughout this application are provided to illustrate the invention.

### Example

The below Example of the present invention shows that a P. emblica-containing composition of this invention confers neuroprotection from cerebral injury from stroke, and/or attenuates cerebral injury from stroke, for instance by increasing the expression of neurotrophic factors such as SDF-1, BDNF, and VEGF; for instance, and without being bound by theory, regulating neuronal growth and axonal regeneration; and upregulating components of the PI3K/Akt/GSK3b pathway; as well as otherwise noted in the below Example and throughout this application. Administration of a P. emblica-containing composition before the induction of a stroke and also after induction of a stroke reduced brain infarct size, rescued mitochondrial functions, and improved functional and neurological outcomes in vivo.

### In-Vivo

1) Animals: Adult male Sprague-Dawley rats, weighing about 240-270 grams, were procured from Zydus Cadila (Ahmedabad, India). All animals were quarantined for 6 days and maintained in cages at room temperature (25±0.5° C.) with a relative humidity (60±5%), 12 hours of light and dark cycle. Water and food were provided ad libitum to animals during the experimental period. All the procedures were conducted after approval and in accordance with strict Institutional Animal Ethics Committee (IAEC) guidelines.

2) Animal grouping and treatment with a P. emblica-containing composition: Animals were divided into 4 groups (n=6).
Group I—“Sham” group—Rats in this group underwent placebo surgery without induction of a stroke. No P. emblica composition according to this invention was administered to this group.
Group II—“Stroke” group—Rats in this group underwent middle cerebral artery occlusion (MCAO) surgery to induce ischemic stroke and then reperfusion. No P. emblica composition according to this invention was administered to this group.
Group III—“Prophylactic” treatment group—Rats in this group underwent middle cerebral artery occlusion (MCAO) surgery to induce ischemic stroke and then reperfusion. Rats were administered Capros® (100 mg/kg) p.o. (orally) daily for 30 days prior to MCAO surgery.
Group IV—“Treatment group”—Rats in this group underwent middle cerebral artery occlusion (MCAO) surgery to induce ischemic stroke and then reperfusion. Rats were administered Capros® (100 mg/kg) 1-hour post-MCAO surgery, specifically one hour after filament removal, during the reperfusion period. Capros® was administered using an oral gavage. Capros® was dissolved in normal saline and administered orally according to the body weight of the animal. Animals were sacrificed post-24 hours of reperfusion and brain samples were collected for further study.

3) Animal surgery:

a) Femoral artery cannulation: Femoral artery cannulation was performed to measure mean arterial blood pressure and analyze various blood gas parameters (Vats, 2019).

Procedure:
1. Rat was anesthetized with isoflurane.
2. The hind limbs were fixed on the table with tape.
3. The fur near inner leg region was shaved, and the skin was cleaned with 0.5% Betadine (iodopovidone) and 70% alcohol.
4. An incision was made on thigh to expose the femoral artery, then muscle attachments were removed using a cotton tip. The femoral artery was isolated from femoral vein and femoral nerve using fine forceps. The artery was ligated with a 3-0 silk suture at the one end and a loose knot on the other end.
5. Then a fine cut was made between the tight knot and loose knot. Holding a PE-50 catheter in place, PE-50 tubing was advanced through the blunt end into the animal's femoral artery at an angle of about 5° to 10°.
6. Successful entry in the artery was verified by observing the flow of blood into the tubing. After observing blood flow into the PE-50 catheter, the silk threads on the artery were tightened over the catheter to keep the catheter in the artery. The needle was pulled out of the PE-50 catheter and blood collected in a heparinized capillary tube for blood gas analysis.

b) Laser Doppler Flowmetry Cerebral Blood Flow (CBF) monitoring: Laser Doppler Flowmetry (LDF) allows continuous measurement of blood flow in tissue samples. A successful MCAO surgery will result in an easily marked reduction in blood flow. LDF measurement is reliable and is economical for use without requiring the need for harvesting of tissue. This technique uses the Doppler shift of laser light reflected by passing blood to produce a normalized blood flow measurement. Blood flow values from LDF outputs are expressed in terms of perfusion units (PFU) which indicate a relative rather than absolute cerebral blood flow (Saraf et al., “Intra-arterial stem cell therapy modulates neuronal calcineurin and confers neuroprotection after ischemic stroke” International Journal of Neuroscience 1-10 (2019a)).

Procedure:
The left scalp was opened, and the skull was exposed with a 2-mm burr hole drilled on the left sphenoid bone (0.5 mm anterior; 6 mm lateral to bregma). The dura was kept intact. The Doppler probe (AD Instruments, Dunedin, New Zealand) was placed above the dura and blood flow through the cortical branch of the MCA (Middle Cerebral Artery) was monitored. CBF was measured in terms of perfusion units (PFU). LDF signals were recorded prior to, during, and after the suture insertion. Rats not exhibiting 70% reduction in cerebral blood flow were excluded from the study.

c) Middle cerebral artery occlusion (MCAO):

Transient focal cerebral ischemia was induced by MCAO using the filament model as previously described (Yavagal et al., “Efficacy and dose-dependent safety of intra-arterial delivery of mesenchymal stem cells in a rodent stroke model” PloS one 9 (2014)).

Procedure:
1. Adult male Sprague Dawley rats were made to fast overnight but free access to water was allowed.
2. Rats were anesthetized with isoflurane. Hair over the neck and groin area was removed.
3. A temperature probe was inserted into the rectum for maintaining body temperature at 37° C.
4. A PE 50 catheter was inserted into the femoral artery for periodic blood sampling for pH, arterial gases and plasma glucose.
5. Then a midline neck incision was made.
6. Carefully the common carotid artery (CCA) was isolated from the surrounding muscles and nerves followed by temporary blockage with a ligature.
7. The external carotid artery (ECA) was isolated, and then an ECA stump prepared by placing two ligatures under the ECA.
8. Occipital artery was ligated and cut from the ECA. The internal carotid artery (ICA) was exposed to see the middle cerebral artery.
9. The CCA and ICA were clipped using microvascular clips.
10. A small incision was made in the ECA between the two ligatures and a silicon coated filament was introduced and advanced up to the bifurcation of the CCA. The filament was passed via the CCA bifurcation into the ICA until it stopped at the microvascular clip. The filament was then fixed in position by tying up the silicon filament over the ICA. The distal part of the ECA was then cut.
11. The microvascular clips were removed, and the filament was advanced through the ICA toward the origin of the MCA. The correct suture position was confirmed by feeling resistance during filament insertion or by advancing the filament a defined distance according to the animal's body weight from the CCA bifurcation.
13. After 90 min of MCAO, the filament was withdrawn to restore the ICA-MCA blood flow.
14. The incision was closed, and rats were returned to their cages, and provided with free access to food and water.

d) Post-Operative Care
Rats were allowed to recover from anesthesia in the lab and were periodically observed for 24 hours post-operatively. After surgery, they were monitored twice a day until sacrificed. Despite undergoing invasive surgical procedures, animals did not display indications of distress following the surgery. Analgesic (Diclofenac sodium) was administered twice a day post-surgery. Animals were observed for 24 hours following all surgeries and then sacrificed. In case the animal displayed signs of hypothermia, especially after induction of cerebral ischemia, animals were protected, in the first 4 hours, by placing them under a heating lamp. Thereafter, animals were returned to their cages with free access to food and water. An intra-peritoneal injection of 0.9% sterile saline solution was given in case of dehydration (indicated by a decrease in skin turgor). (Pravalika et al., 2019).

4) Neurodeficit scoring: Neurological scores were derived on 12 points which measure sensory, motor, and balance impairment. The entire scoring is divided into 4 main sections: postural reflex, visual placing, tactile placing and proprioception. Scores were given on the following basis:
a) Postural reflex
b) Visual placing
   a. Forward
   b. Sideways
c) Tactile placing
   a. Dorsal surface of paw
   b. Lateral surface of paw
d) Proprioceptive placing
   For postural reflex, a score of 0 was given when no observable deficit was seen, 1 for limb flexion during hang test and 2 for lateral push deficit. For placing tests, a score of 0 was given for complete immediate placing, 1 for incomplete or delayed placing (<2 seconds) and 2 absence of placing.

The summation of the neurobehavioral scores attained after testing for each scale were used to denote the degree of neurological deficit. Importantly, rats not exhibiting neurological score, having small infarct sizes and inconsistent physiological parameters were excluded from the study. (Ley et al., “Stilbazulenyl nitrone, a second-generation azulenyl nitrone antioxidant, confers enduring neuro protection in experimental focal cerebral ischemia in the rat: neurobehavior, histopathology, and pharmacokinetics” The Journal of Pharmacology and Experimental Therapeutics 313:1090-1100 (2005)).

5) Rotarod test: For evaluating motor function, rotarod test was performed. The rats were placed on the rotarod cylinder (RotaMex, Columbus Instruments, Columbus, Ohio) and latency to fall (sec) was recorded. The speed was gradually increased from 10 to 20 rpm over 5 minutes. The trial ended if a rat fell off the device or if it spun around for 2 consecutive revolutions without the rat attempting to walk. The cut-off time was set to 180 seconds. The rats were initially trained on the rotarod cylinder for 3 consecutive days before undergoing the MCAO procedure (Bhattacharya et al., 2013).

6) Grip strength: Rat was placed on a grid and was allowed to grab the grid with both fore paws and then was pulled by holding the tail and the maximum force (g) required to hold the grid was measured (ALMEMO Measuring Instruments, Ahlborn Mess- and Regelungstechnik GmbH, Holzkirchen, Germany). (Shen et al., “Characterization of endogenous neural progenitor cells after experimental ischemic stroke” Current Neurovascular Research 7:6-14 (2010)).

7) TTC staining: Staining with TTC (Triphenyl tetrazolium chloride) (Sigma-Aldrich, St. Louis, Mo.) is a rapid method to assess infarct size in rat brains after stroke. TTC is a white or faint yellow powder and is colorless in solution. When TTC comes in contact with rapidly respiring tissues, it takes up electrons from the mitochondrial ETC (electron transport chain) resulting in the reduction of the colorless stain to a deep pink/red formazan compound. The intensity of the red color is proportional to the rate of respiration in those tissues. An infarct region having less mitochondrial activity does not convert TTC and remains unstained (Vats et al., 2019).

After neurological examination, rats were sacrificed by cervical dislocation and the brain was isolated in chilled ice. Six coronal sections 2 mm thick were taken using brain matrix. These sections were then incubated in 0.1% TTC (PBS) at 37° C. for 30 min. Viable brain sections are stained brick red with TTC, whereas an infarcted/non-viable region remains unstained.

8) Tissue lysate preparation: The rats were sacrificed under light anesthesia after 24 hours by cervical decapitation, and the whole brain was collected. The cerebellum was rapidly removed from the whole brain tissue and the remaining brain was rinsed with ice-cold 0.9% NaCl and finally ipsilateral cortex was separated. The cortex was used to prepare brain homogenate by using ice-cold extraction lysis buffer/RIPA lysing buffer (prepared in-house) in a homogenizer. Then the homogenate was sonicated and centrifuged at 12,000 rpm for 20 min at 4° C. to remove cellular debris, and the supernatant was used for the determination of GSH (glutathione), Nitrite, and TBARS (thiobarbituric acid reactive substances) activities. Supernatant obtained from this extraction procedure was stored at −80° C. and used for western blotting. The protein concentration was determined by using BSA (Pravalika et al., 2019).

9) Determination of protein by BCA reagent: The protein concentration of sample was determined by BCA (bicinchonic acid) assay (Pierce BCA Protein Assay Kit, Thermo Fisher Scientific, Waltham, Mass.). Working BCA reagent comprises of BCA reagent A and reagent B (50:1). Sample dilution (50 times) was prepared and from it 25 μl of sample was added to 200 μl of working BCA reagent in 96 well plate. Working reagent with water instead of sample was used as blank. The plate was incubated for 30 minutes at 37° C. The absorbance was taken at 562 nm. The amount of protein was calculated by plotting standard curve of Bovine serum albumin (BSA). (Saraf, 2019b).

10) Determination of GSH levels by DTNB (5,5-dithio-bis-(2-nitrobenzoic acid)) assay: 100 μl of sample was mixed with 100 μl of Ellman's reagent (HiMedia Laboratories, Mumbai, India) in 0.1M phosphate buffer (pH 8.0). The mixture was then incubated for 10 min at 38° C. in a water bath. Absorbance was measured at 412 nm using a micro plate reader. Amount of GSH present in the cells was calculated by plotting a standard curve of glutathione. (Vats et al., 2019).

11) Determination of nitrite levels by Griess method: 100 μl of sample was added in a 96 well plate, to which 100 μl of working Griess reagent (Sigma-Aldrich, St. Louis, Mo.) and 50 μl of water was added and incubated for 30 min at room temperature. Absorbance was measured at 540 nm. Sodium nitrite solution was used as standard (Pravalika et al., 2019).

12) Determination of MDA levels by TBA assay: MDA (malondialdehyde, an indicator of lipid peroxidation) (Sigma-Aldrich, St. Louis, Mo.) was estimated by TBA assay. MDA was estimated by adding 100 μl sample, 100 μl sodium dodecyl sulphate (SDS), 750 μl thiobarbituric acid (TBA), 300 μl water and 750 μl acetic acid to a 2 ml centrifuge tube. The above mixture was placed in water bath for 1 h at 95° C. after which 250 μl of the mixture was added to a 96 well plate and absorbance was taken at 532 nm using a microplate reader. The levels of MDA were determined using MDA as a standard (Saraf et al., 2019b).

13) Isolation and characterization of intact mitochondria in the rat brain: Rat brain mitochondria were isolated according to the protocol described by Amigo et al. (“Isolating Brain Mitochondria by Differential Centrifugation” Bio-protocol 6:e1810 (2016)) with a small modification which allowed to obtain mitochondria with much better functional characteristics.

Procedure:
1. The rat was anesthetized using isoflurane and sacrificed by cervical dislocation, immediately after the complete brain was removed and placed in an ice-cold beaker with chilled extraction buffer (125 mM sucrose, 250 mM mannitol, 10 mM HEPES, 10 mM EGTA, 0.01% BSA, 1× protease inhibitor; all products from Sigma-Aldrich (St. Louis, Mo.)).
2. The brain was rinsed to remove blood by adding and removing cold fresh buffer, until most of the blood was removed (5-6 washes).
3. The brain was minced with the help of small scissors in the beaker.
4. The minced brain was transferred into a Dounce homogenizer with 3 ml of cold extraction buffer.
5. The homogenizer was placed in an ice container, the tissue was then homogenized ten times with A pestle (looser) and another ten times with B pestle (tighter). Bubble formation was avoided to attain mitochondria of high quality.
6. The homogenate was collected and transferred to a centrifuge tube, followed by performing differential centrifugation.
7. Initially the homogenate was centrifuged for 10 min at 700×g and 4° C. The supernatant was collected in a new ice-cold tube and pellet containing nuclei and intact cells were discarded.
8. The supernatant from the above step was again centrifuged at 700×g for 10 min at 4° C. The pellet obtained was discarded and the supernatant obtained was combined with the supernatant of the previous step and centrifuged at 10,000×g for 15 min at 4° C.
9. The supernatant obtained from the previous step was discarded and the pellet was re-suspended in ice-cold extraction buffer with 0.02% digitonin. (Sigma-Aldrich, St. Louis, Mo.).
10. The re-suspended pellet was centrifuged at 10,000×g for 15 min at 4° C. The pellet containing mitochondria obtained from the step was resuspended in 0.1 ml of extraction buffer.
11. Protein concentration was determined by BCA method.
12. The quality and intactness of isolated mitochondria was determined by Western blotting by checking mitochondrial membrane protein (TOMM20) (Abcam, Cambridge, Mass.) expression.

14) Mitochondrial complex assays: Measurement of the chain complexes is very important to understand the mitochondrial dysfunction in ischemic stroke. The activity of complexes I, II and IV in mitochondria isolated from rat brain was measured.

Complex I: The first complex of the oxidative phosphorylation system within the mitochondria is complex I, also called NADH dehydrogenase. It acts as the port of entry of electrons into the respiratory chain following oxidation of NADH and electron transport to coenzyme-Q. It is the largest among all the complexes of the mitochondria. A deficiency of complex I is probably the most frequently encountered cause of mitochondrial disease. The activity of complex I was assayed by means of spectrophotometry. The oxidation of NADH at 550 nm in a mitochondria-enriched brain tissue homogenate was measured (Dave et al., “Ischemic preconditioning targets the respiration of synaptic mitochondria via protein kinase Cε. Journal of Neuroscience 28:4172-4182 (2008)).

Procedure:
1. Complex I specific activity was measured by following the decrease in absorbance due to oxidation of NADH at 550 nm.
2. The assay mixture contained 0.2 M glycyl glycine, 6 mM NADH, 1.05 mM cytochrome-c and 0.02M sodium bicarbonate.
3. In a 96 well plate 35 μl of glycyl glycine (0.2M) was added then 10 μl of cyt-c, followed by 10 μl of NADH then 0.24 ml double distilled water was added.
4. Thereafter 1 μl of sample was added and onto that 2 μl sodium bicarbonate (0.02M) was added.
5. The reaction was measured by change in OD at 550 nm for 180 sec.
6. The activity is expressed as nanomole of NADH oxidized per minute per milligram of mitochondrial protein.

$$Calculation = \frac{Change in OD / min \times 0.262 \times 3 \times 1000}{mg protein in assay volume i . e . 1 μl}$$

Complex II: A variable proportion of mitochondrial complex II in an isolated sample is inactive due to tight binding of oxaloacetate, a competitive inhibitor. It is essential to ensure that the enzyme is fully activated, and this can be achieved by pre-incubation with succinate. The activity of complex II is also dependent on the disruption of the inner mitochondrial membrane (Dave et al., 2008).

Procedure:
1. The assay was done in a 96 well plate. 150 μl of (0.2M) sodium phosphate buffer was added, then 20 μl (0.6M) Succinate, followed by 30 ul bovine serum albumin, then 25 ul of 0.03M potassium ferricyanide (freshly prepared) was added, followed by 175 μl of DDW and then finally 2.5 μl of sample was added.
2. Change in OD was observed at 420 nm for 180 seconds and the activity was expressed in n moles of substrate/min/mg protein.

$$Calculation = \frac{Change in OD / min \times assay volume \times 0.435 \times 106}{mg protein in assay volume i . e . 10 μl \times 1000}$$

Activity expressed as nanomole of substrate per minute per milligram of mitochondrial protein.

Complex IV: Complex IV activity was assessed by evaluating the oxidation of cytochrome c (II) at 550 nm. The reaction buffer contained 0.075M sodium phosphate buffer pH 7.4 and 0.3 mM cytochrome-c (reduced).

Procedure:
1. In a 96 well plate 25 μl reduced cytochrome-c was added, then 175 μl of phosphate buffer was added, onto that 2.5 μl of sample was added, and change in OD was measured at 550 nm for 180 seconds. Activity expressed as nanomole per minute per milligram of mitochondrial protein.

$$Calculation = \frac{Change in OD / min \times assay volume \times 3 \times {10}^{6}}{mg protein in assay volume i . e . 2.5 μl \times 60 \times 29.5}$$

15) Mitochondrial respiration studies: Mitochondrial respiration studies were performed on High-Resolution Respirometry Oxygraph-2K (Oroborus Instruments, Innsbruck, Austria). The chambers were prepared by cleaning with water and ethanol. Respiration medium was added to each of the chambers and air calibration was performed. The system was allowed to stabilize until a stable oxygen flux was obtained. 300 μg of freshly isolated mitochondria were then added into the chambers. This was followed by addition of complex I substrates, 5 mM pyruvate, 5 mM malate and 410 mM glutamate to stimulate mitochondrial respiration. 1 mM ADP was added to induce OXPHOS (oxidative phosphorylation). 10 mM succinate was added to induce C-I+C-II respiration. 5 mM oligomycin was then added to inhibit ATP synthase and get leak respiration state. Maximum uncoupled respiration was obtained following addition of FCCP, an uncoupler of mitochondrial oxidative phosphorylation. Complex I and III were inhibited by addition of 0.5 uM rotenone and 2.5 uM antimycin A, respectively. (Maiti, “The role of caseinolytic mitochondrial matrix peptidase proteolytic subunit (CLPP) in regulation of mitochondrial ribosome biogenesis in mammals” Doctoral Dissertation, Faculty of Mathematics and Natural Sciences, University of Cologne, Germany (2015)). State 3 and state 4 respiration was determined and the respiratory control ratio (RCR) was calculated. (Pesta and Gnaiger, “High-resolution respirometry: OXPHOS protocols for human cells and permeabilized fibers from small biopsies of human muscle” Methods Mol. Biol. series, Mitochondrial Bioenergetics 810:25-58 (Springer 2012)).

$$RCR = \frac{State 3 respiration}{State 4 respiration}$$

16) Western blotting: For Western blotting, 30 μg of tissue lysate was denatured in gel loading buffer (100 mM Tris-HCL (pH 6.8), 2% sodium dodecyl sulfate, 20% glycerol and 0.2% bromophenol blue) in dry bath at 95° C. for 5 min. The samples were loaded on 10% SDS-polyacrylamide gel along with ladder. Electrophoresis was carried out in gel running buffer containing 250 mM glycine, 25 mMTris, and 0.1% SDS. After electrophoresis, proteins were transferred onto a PVDF membrane in trans-blotting system for 45 minutes with constant power supply of 50 V. After transfer, the membrane was blocked in 3% BSA for 2 hours. The blot was then incubated with primary antibodies (Abcam, Cambridge, Mass.) of different proteins overnight at 4° C. After three washes with TBST (TBS+0.05% Tween-20) for 5 min each, the blot was incubated with HRP (horseradish-peroxidase)-conjugated goat anti-rabbit/goat anti-mouse secondary antibodies. After washing with TBST, proteins were revealed with ECL (enhanced chemiluminescence) (BioRad, Hercules, Calif., USA) and their expression level was measured by densitometry. GAPDH and β actin were used as control for immunoblotting. Band density values were normalized to GAPDH and β-actin (Vats et al., 2019).

Results:

1) Laser Doppler Flowmetry

During MCAO surgery, cerebral blood flow (CBF) was monitored by Laser Doppler Flowmetry to confirm middle cerebral artery occlusion and interruption of cerebral bloodflow (induction of stroke). A baseline for 5 minutes was initially taken before commencement of the surgery. As shown in FIG. 1, during filament insertion, a decline of about 70-75% in CBF was markedly observed and recorded. This pattern was observed during the 90 minutes of occlusion. A robust reperfusion peak was observed after retracting the filament post 90 minutes.

2) Blood Gas Parameter

Physiological parameters such as pO₂, pCO₂, and pH were recorded throughout the surgery. Rectal temperature was maintained at around 37±0.5° C. and blood glucose at 80-120 mg/dl during surgery.

TABLE 1
Monitoring of physiological parameters during surgery

| Parameters | | Post ischemia | Post ischemia |
|---|---|---|---|
| monitored | Pre-ischemia | (45 minutes) | (115 minutes) |
| pH | 7.32 ± 0.005 | 7.18 ± 0.001 | 7.163 ± 0.001 |
| pO₂ (mmHg) | 107 ± 2 | 95.3±3 | 100 ± 2 |
| pCO₂ (mmHg) | 48.1 ± 2 | 51.4±2 | 54 ± 2 |

3) Rotarod

FIG. 2 shows the effect of Capros® on rotarod performance by rats 24 hours following cerebral ischemia (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤0.001). Retention times on the rotarod were measured at 3 different speeds (5 rpm, 10 rpm, 20 rpm).

It was observed that the latency to fall off the rotarod significantly decreased in Stroke induced rats when compared with the Sham group. The administration of Capros® to rats in both Prophylactic and Treatment groups significantly increased the latency to fall off the rod (i.e., increased retention time on the rotarod) when rats were made to run at a rotarod speed of 5 rpm. At a rotarod speed of 10 rpm, the administration of Capros® prior to surgery significantly increased the latency of rats in the Prophylactic group to fall off the rod, as compared with Stroke rats, and accordingly acted on the treated subjects, providing neuroprotection from brain injury and attenuating injury such as impairment of balance from stroke in the brain in animals administered an effective amount of the P. emblica extract Capros®.

4) Grip Strength

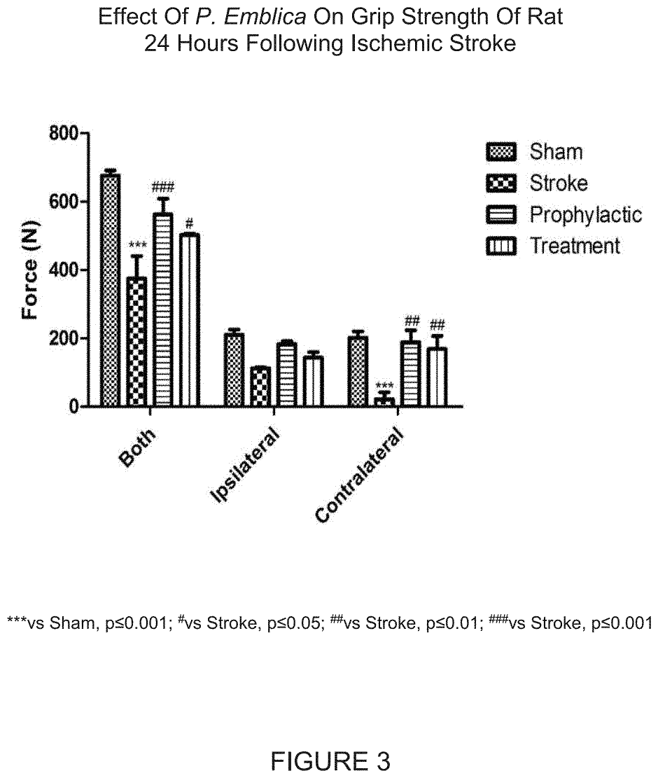

FIG. 3 shows the effect of Capros® on grip strength of rat 24 hours following cerebral ischemia (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤0.001).

It was observed that the grip strength of the animal decreased in the Stroke group after ischemic insult when compared to Sham. This decrease was also significant in the contralateral side of the animal. P. emblica-administered Prophylactic and Treatment groups displayed a significant improvement in the grip strength in comparison to Stroke animals, which underwent ischemia without P. emblica administration. This improvement was also significant in the contralateral side of the animal. Accordingly, FIG. 3 shows that the administration of an effective amount of the P. emblica extract Capros® acted on the treated subjects, providing neuroprotection from brain injury, and attenuating brain injury such as impairment of motor function from stroke in the brain, as compared with rats not administered the composition.

5) Neurodeficit Scoring

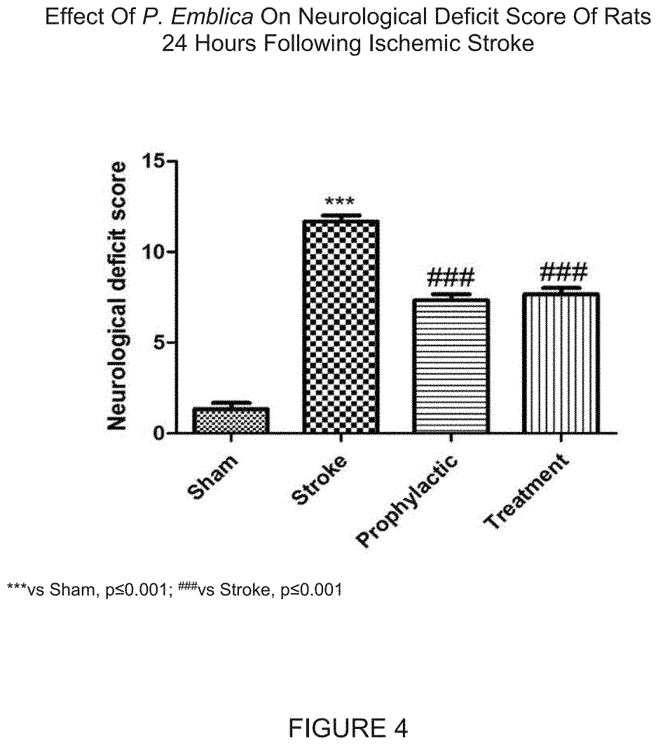

FIG. 4 shows the effect of Capros® on the neurological deficit score of rats 24 hours following cerebral ischemia (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤001). Neurological function was assessed prior to ischemia and 1 day after MCAO. Rats administered Capros® (both Prophylactic and Treatment groups) showed improvement in the neuro-deficit scores in comparison to Sham and Stroke animals. Accordingly, FIG. 4 shows that the administration of an effective amount of the P. emblica extract Capros® acted on the treated subjects, providing neuroprotection from brain injury, and attenuating brain injury from stroke such as impairment of sensory function, motor function, and/or balance in the brain, as compared with subjects not administered the composition.

6) TTC Staining

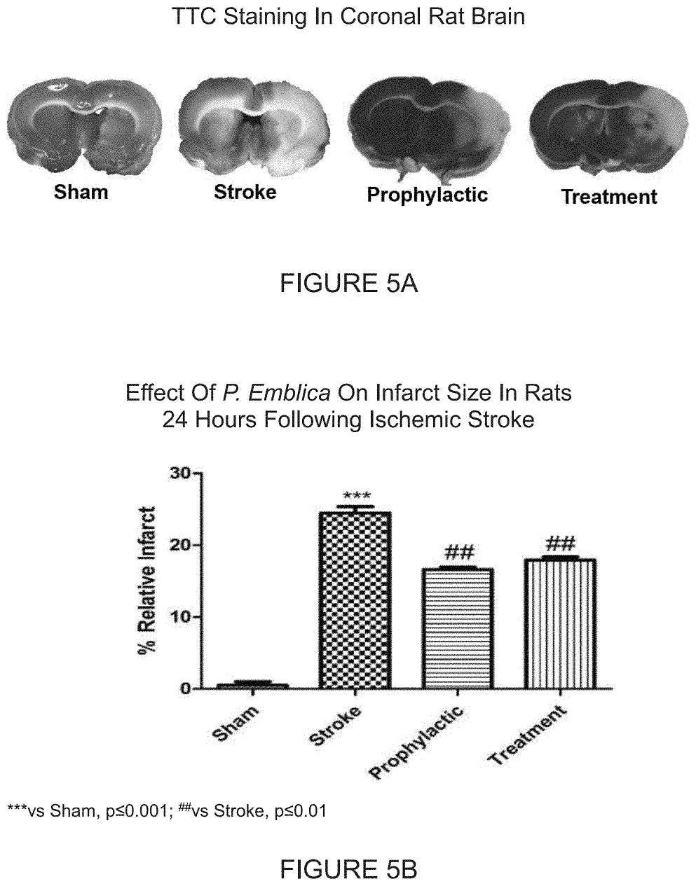

TTC staining in coronal rat brain was performed as described above and shown in FIG. 5A. The photomicrograph of the Sham rat brain shows consistently red (dark) staining, indicating little to no infarct. The photomicrograph of the Stroke rat brain shows a substantial infarct (area with little to no dark staining at right of picture), and the photomicrographs of the Prophylactic and Treatment rat brains showed the formation of a small infarct (area with little to no dark staining at right of each picture).

FIG. 5B is a chart showing the effect of Capros® on infarct size in rats post 24 hours of cerebral ischemia (*vs Sham, p≤0.05; **vs Sham, p≤0.01;***vs Sham, p≤0.001; vs Stroke, p≤0.05; ^{##}vs Stroke, p≤0.01; ^{###}vs Stroke, p≤0.001). Capros® significantly reduced infarct size in the Treatment and Prophylactic groups in comparison with the Stroke animals. Accordingly, FIGS. 5A and 5B show that the administration of an effective amount of the P. emblica extract Capros® acted on the treated subjects, providing neuroprotection from brain injury, and attenuating infarct size in the treated animals, compared with subjects not administered the P. emblica composition.

7) Biochemical Assays

### a) GSH Assay

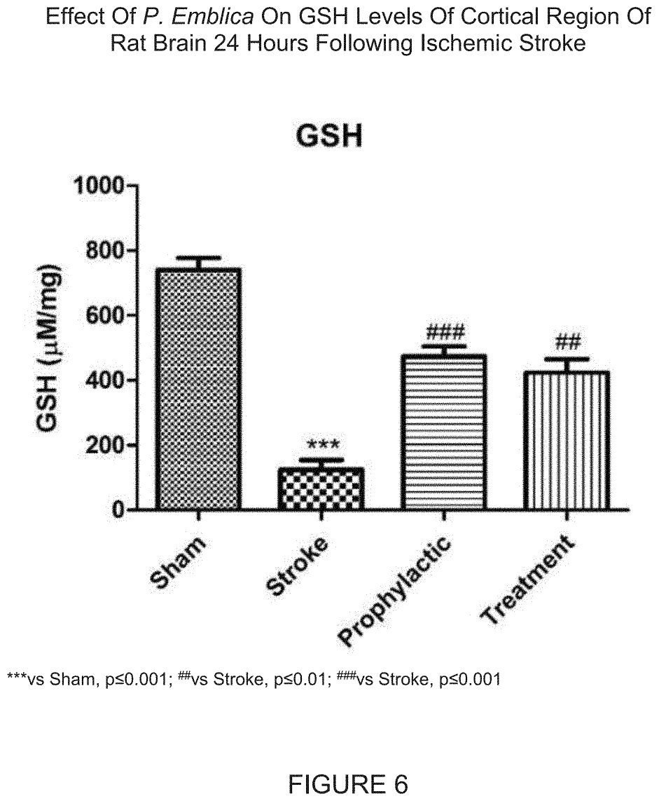

FIG. 6 shows the effect of Capros® on GSH levels of cortical region of rat brain 24 hours following cerebral ischemia (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤0.001).

The amount of GSH was significantly decreased 24 hours after stroke in Stroke animals, in comparison with Sham animals. Capros® significantly increased the levels of GSH seen in Treatment and Prophylactic groups in comparison to Stroke animals. Accordingly, FIG. 6 shows that the administration of an effective amount of the P. emblica extract Capros® acted on the treated subjects, providing neuroprotection from brain injury, and attenuating brain injury such as related to GSH depletion from stroke, as compared with subjects not administered the composition.

### b) Nitrite Assay

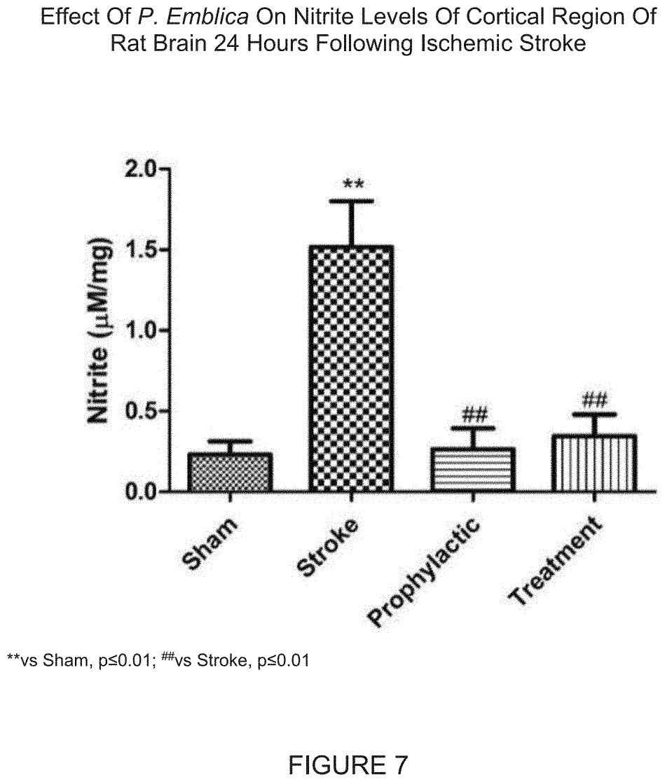

FIG. 7 shows the effect of Capros® on nitrite levels of cortical region of rat brain 24 hours following cerebral ischemia (*vs Sham, p≤0.05; **vs Sham, p≤0.01;***vs Sham, p≤0.001; vs Stroke, p≤0.05; ^{##}vs Stroke, p≤0.01; ^{###}vs Stroke, p≤0.001).

Nitrite levels were significantly increased 24 hours following cerebral ischemia in Stroke animals, as compared with Sham animals with no cerebral ischemia. Capros® significantly decreased the nitrite levels in Treatment and Prophylactic groups in comparison to the Stroke animals. Accordingly, FIG. 7 shows that the administration of an effective amount of the P. emblica extract Capros® acted on the treated subjects, providing neuroprotection from brain injury, and attenuating brain injury such as related to increased nitrite levels/nitrosative stress from stroke, as compared with subjects not administered the composition.

### c) MDA Assay

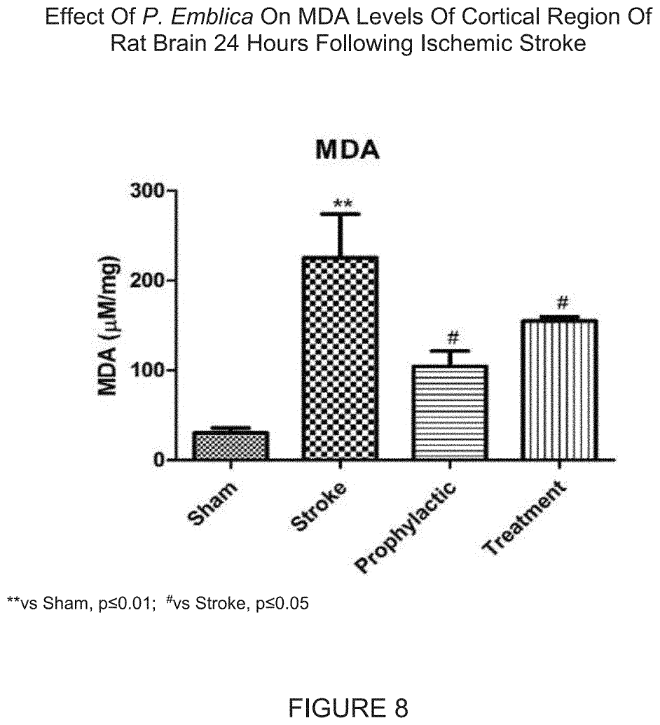

FIG. 8 shows the effect of Capros® on MDA levels of cortical rat brain 24 hours following cerebral ischemia. (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤0.001).

MDA levels were significantly increased 24 hours following cerebral ischemia in Stroke animals, as compared with Sham animals with no cerebral ischemia. Similar to findings regarding nitrite levels, Capros® significantly decreased MDA levels in Treatment and Prophylactic group animals as compared with Stroke animals. Accordingly, FIG. 8 shows that the administration of an effective amount of the P. emblica extract Capros® acted on the treated subjects, providing neuroprotection from brain injury, and attenuating brain injury such as related to increased oxidative stress from stroke, compared with subjects not administered the composition.

8) Characterization of Isolated Mitochondria

Isolated mitochondria were assessed for their integrity. Isolated mitochondria from each group expressed TOMM20, which is a marker for the mitochondrial outer membrane, confirming the integrity of the mitochondria.

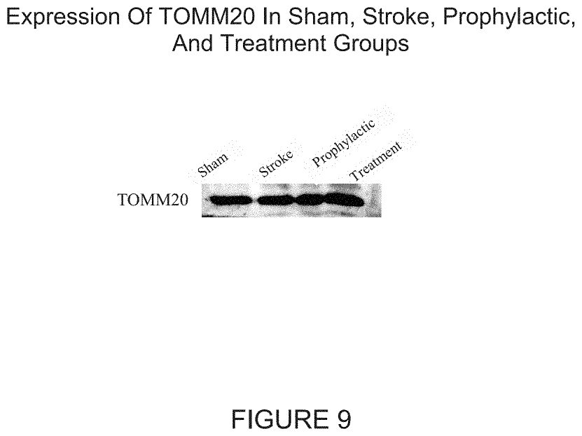

FIG. 9 shows a Western blot representing the expression of TOMM20 protein in Sham, Stroke, Prophylactic and Treatment groups.

### Complex I Activity

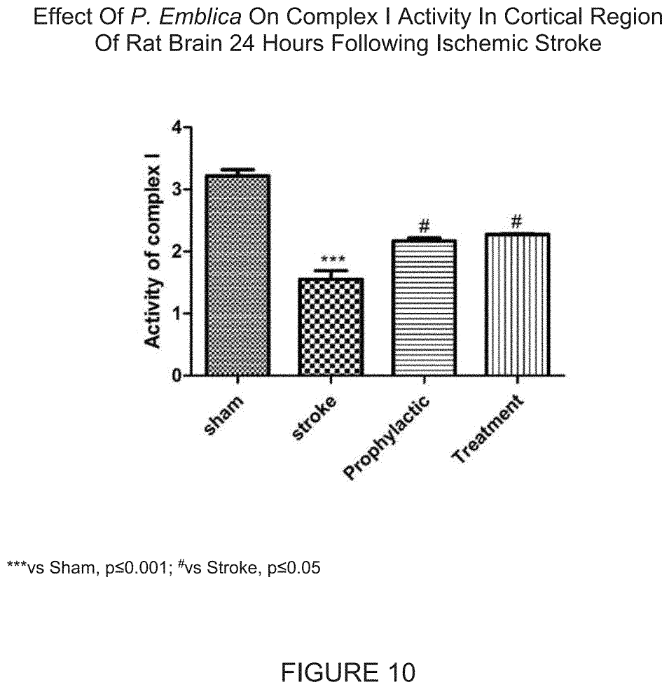

FIG. 10 shows the effect of Capros® on complex I activity in cortical region of rat brain post-24 hours following cerebral ischemia. (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤0.001).

Complex I activity was estimated in isolated mitochondria from brains of Sham, Stroke, Prophylactic, and Treatment groups. Significant reduction in complex I activity was observed following induction of cerebral ischemia in Stroke animals as compared with Sham animals. Both Prophylactic animals and Treatment animals, having been administered Capros®, demonstrated significant improvement in the activity of complex I compared with Stroke animals that did not receive Capros®. Accordingly, FIG. 10 shows that the administration of an effective amount of the P. emblica extract Capros® acted on the treated subjects, providing neuroprotection from brain injury, and attenuating brain injury such as related to mitochondrial dysfunction from complex I activity from stroke, as compared with subjects not administered the composition.

### Complex II Activity

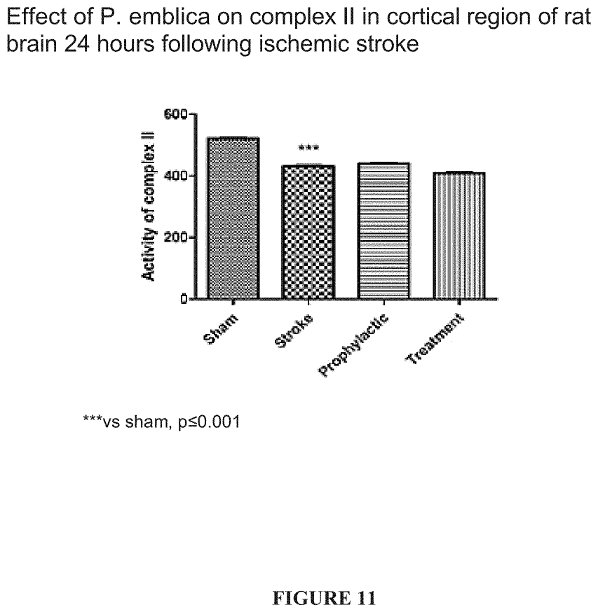

FIG. 11 shows the effect of Capros® on complex II in cortical region of rat brain 24 hours following cerebral ischemia (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤0.001). Significant reduction in mitochondrial complex II activity was observed following induction of stroke (cerebral ischemia) in Stroke animals, as compared to the Sham group. The administration of Capros® in the stated dosage to Treatment animals and Prophylactic animals did not improve the activity of complex II.

### Complex IV Activity

FIG. 12 shows the effect of Capros® on complex IV of cortical region of rat brain 24 hours following cerebral ischemia (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤0.001).

Significant reduction in mitochondrial complex IV activity was observed following induction of stroke (cerebral ischemia) in Stroke animals, as compared to the Sham group. Capros® significantly improved mitochondrial complex IV activity in Treatment and Prophylactic animals, as compared to Stroke animals, which were not administered Capros®. Accordingly, FIG. 12 shows that the administration of an effective amount of the P. emblica extract Capros® acted on the treated subjects, providing neuroprotection from brain injury, and attenuating brain injury such as related to mitochondrial dysfunction from complex IV activity from stroke, compared with subjects not administered the composition.

### Mitochondrial Respiration Studies

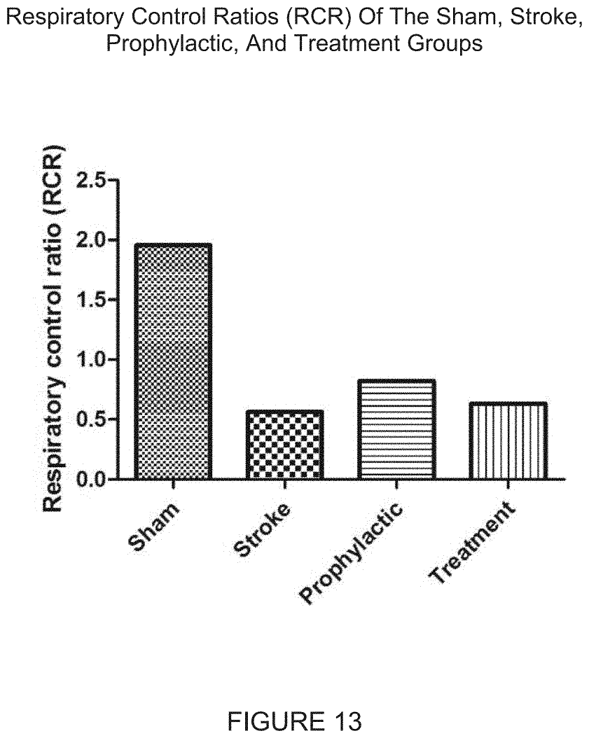

FIG. 13 represents the respiratory control ratio (RCR) between the different groups: Sham, Stroke, Prophylactic, and Treatment. The RCR in Stroke rats decreased as compared to animals in the Sham group. Improvement in mitochondrial respiration in animals of the Prophylactic and Treatment groups following administration of Capros® was observed. The improvement was not found to be significant.

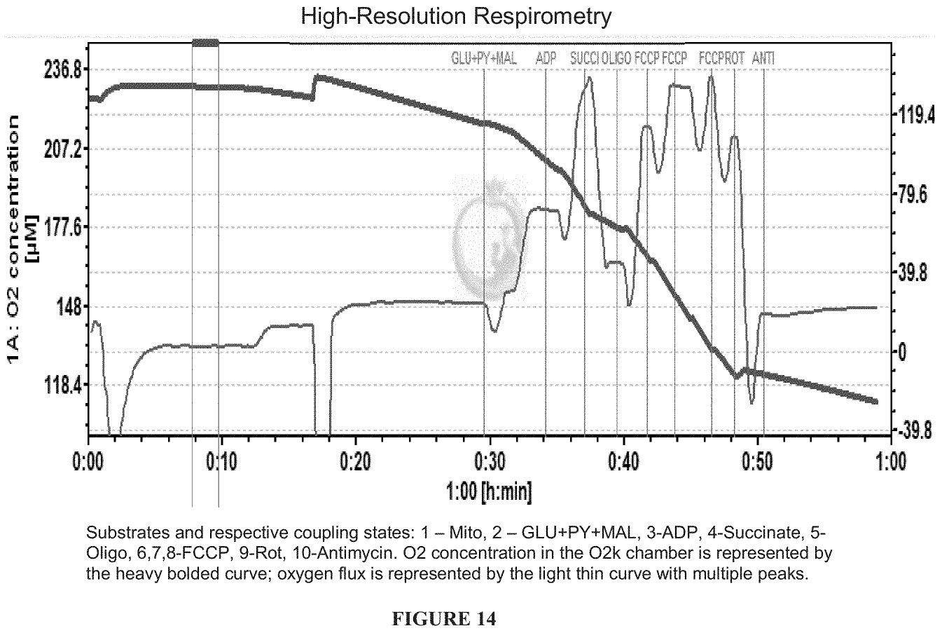

FIG. 14 shows representative respiration by Oroboros High Resolution Respirometry Oxygraph 2K (Oroboros Instruments, Innsbruck, Austria). Substrates and their respective coupling states are identified on the upper horizontal axis (1—freshly isolated mitochondria (“MITO”), 2—Complex 1 substrates glutamate (GLU), malate (MAL), pyruvate (PY) (GLU+PY+MAL), 3—ADP, 4—Succinate (“SUCCI”), 5—Oligomycin (“OLIGO”), 6, 7, 8—FCCP, 9—Rotenone (“ROT”), 10—Antimycin (“ANTI”)). O2 concentration in the O2k chamber is represented by the heavy bolded curve. The light thin curve with multiple peaks depicts the oxygen flux.

### 9) Western Blotting

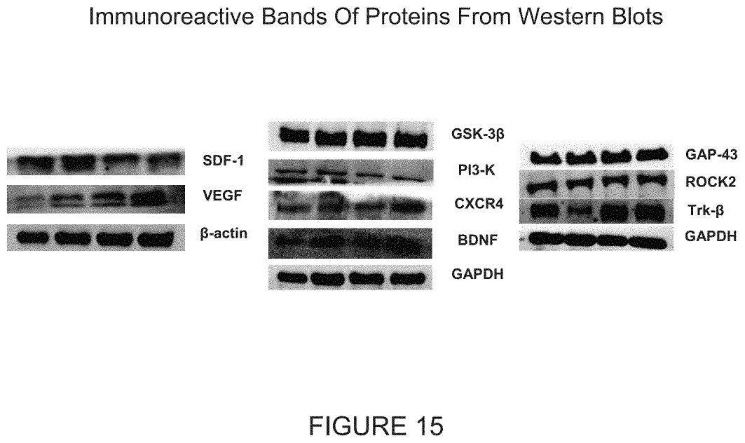

FIG. 15 shows immunoreactive bands on Western blots for various proteins. Western blotting was carried out to check for the effect of Capros® on the expression of various proteins. Immunoblotting was performed for GSK-3β (glycogen synthase kinase-3 beta), PI3-K (phosphatidylinositol-3 kinase), SDF-1 (stromal cell derived factor 1), CXCR4 (chemokine receptor type 4), BDNF (brain derived neurotrophic factor), Trkβ (tyrosine receptor kinase beta), VEGF (vascular endothelial growth factor), ROCK2 (rho-associated coiled-coil containing protein kinase 2) and GAP-43 (growth associated protein-43). GAPDH and β-actin were used as controls.

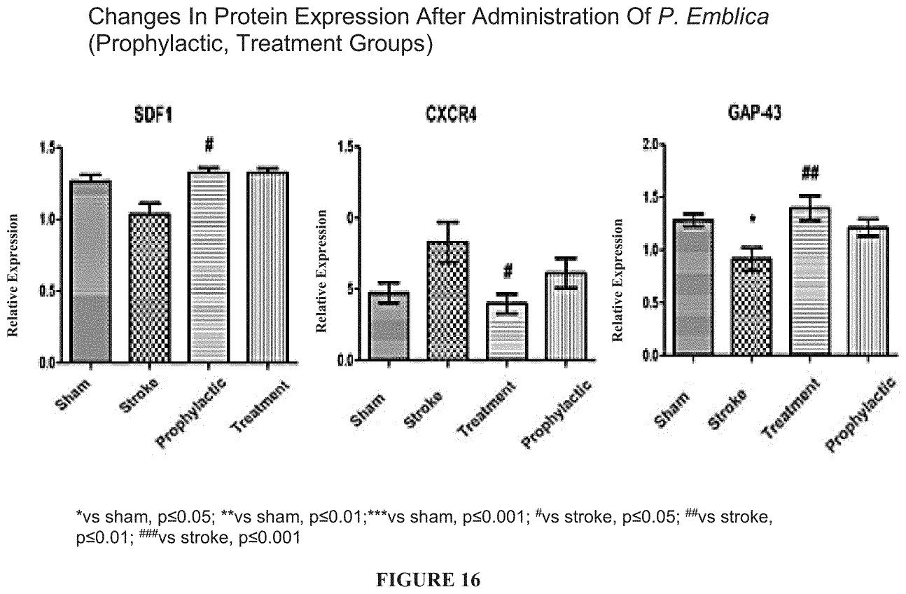

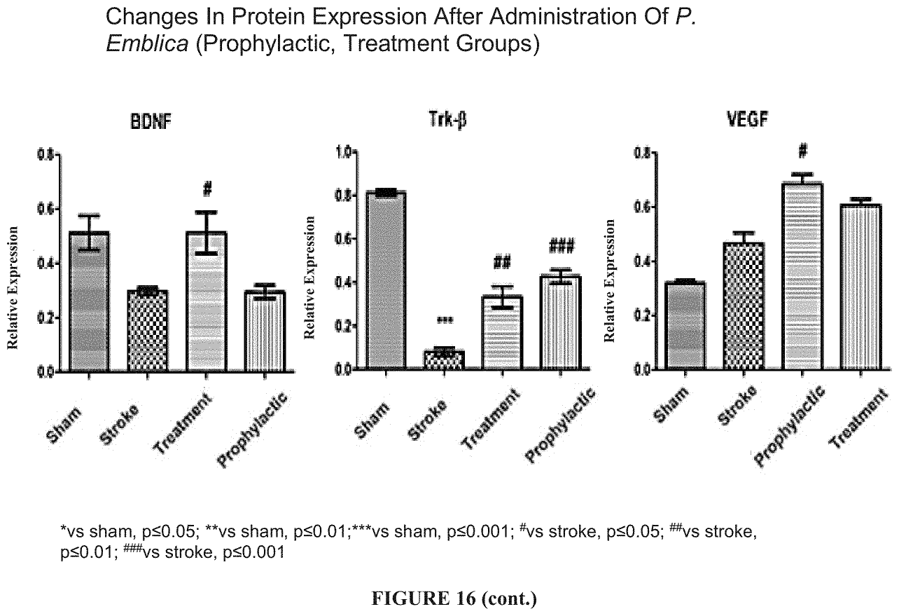

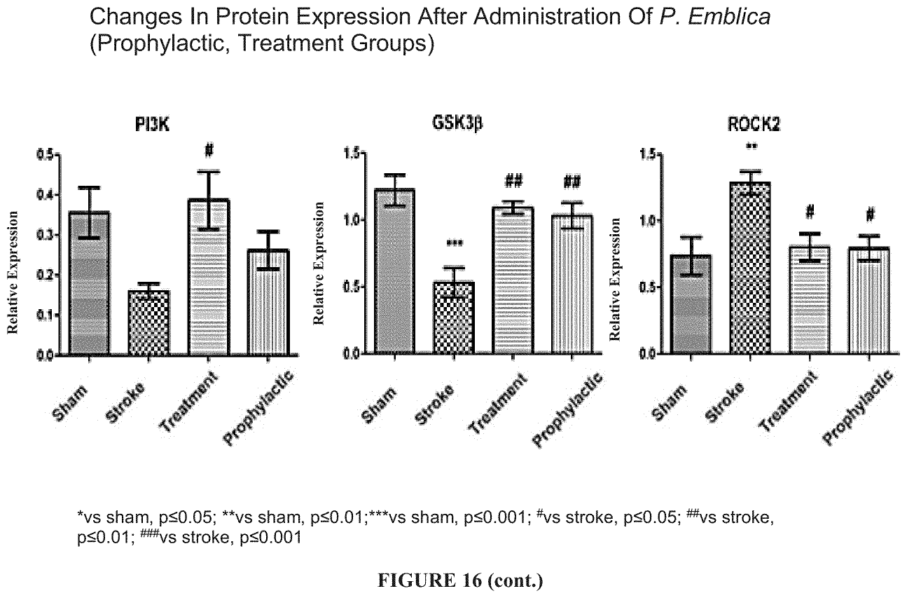

FIG. 16 is a graph representing relative expression of SDF-1, CXCR4, GAP-43, BDNF, Trk-β, VEGF, PI3K, GSK3β, and ROCK2 in Sham and Stroke groups as well as groups administered Capros® (Prophylactic and Treatment) (*vs Sham, p≤0.05; **vs Sham, p≤0.01;***vs Sham, p≤0.001; ^{#}vs Stroke, p≤0.05; ^{##}vs Stroke, p≤0.01; ^{###}vs Stroke, p≤0.001).

### DISCUSSION

A highly desirable goal for acute ischemic stroke therapy is neuroprotection. Protecting the ischemic brain from injury from stroke and also protecting neurons from the detrimental effects of reperfusion is of utmost importance from a therapeutic stand point (Patel and McMullen, “Neuroprotection in the treatment of acute ischemic stroke” Progress in Cardiovascular Diseases 59:542-548 (2017)). Different studies have suggested that components of plant origin are promising and can have an impact on the treatment of neurological disorders (Pravalika et al., 2019). P. emblica is one of those whose medicinal properties are upfront and of paramount medicinal importance. P. emblica fruit extract is reported to contain polyphenolic compounds and vitamins that act as antioxidants and may have a role in making the body defense system robust (Liu et al., “Identification of phenolics in the fruit of emblica (Phyllanthus emblica L.) and their antioxidant activities” Food Chemistry 109:909-915 (2008)). P. emblica has also been shown to target the phosphoinositide 3-kinase/glycogen synthase kinase3β (PI3K/GSK3β) signaling pathway in cardiac ischemia-reperfusion injury. P. emblica is reported to increase the expression of different trophic factors which upon binding to their respective receptors lead to receptor phosphorylation and subsequent activation of PI3K/Akt and other downstream signaling proteins (Thirunavukkarasu et al., 2015).

As shown above, administration of a P. emblica-containing composition, Capros®, as a prophylactic and as a 1-hour post ischemic stroke supplement treatment, elicited significant functional neurological recovery. The neurological deficit caused as a result of ischemic insult was attenuated significantly by Capros® at 100 mg/kg dose in animals with 90 minutes of MCAO occlusion followed by 24 hours of reperfusion as compared to healthy control animals. Capros® at a dose of 100 mg/kg oral treatment 1 hour post stroke significantly reduced the infarct area as compared to Stroke group. Motor impairment is apparent following stroke induction, as evident by reduced retention time on the rotating rod and in grip strength assessment of animals. Capros®, administered prophylactically and as a treatment, was able to improve motor coordination in animals as demonstrated by the significant improvement in the rotarod and grip strength test. Both prophylaxis and treatment with Capros® were effective to a similar extent.

An increase in oxidative and nitrosative stress is observed following cerebral ischemia (Sarmah et al., 2019), and may be measured for instance by an increase in MDA and nitrite levels. Following MCAO, Stroke animals demonstrated increased nitrite and MDA levels, while reduction in GSH levels were seen. Treatment and prophylaxis with Capros® reduced and normalized the elevated nitrite and MDA levels. Elevation in the levels of GSH were also observed in post-stroke animals which were treated with Capros® or were given Capros® prophylactically. Without being bound by theory, the antioxidant effects exerted by Capros® via the inhibition of lipid peroxidation and free radical scavenging may be one among all possible neuroprotective mechanisms against cerebral ischemia.

Mitochondrial dysfunction post ischemia exacerbates ischemic damage in the brain (Sarmah et al., 2019). Re-establishing circulation after a period of blockage results in a surge in oxygen concentration leading to excessive production of free oxygen radicals from the mitochondria (Pravalika et al., 2019). This phenomenon of ischemic reperfusion injury is highly detrimental to neurons. The mitochondrial respiratory chain generates a continuous flux of oxygen radicals. It has been estimated that ≈2% of the oxygen reacting with the respiratory chain leads to formation of superoxide radical. The effect of oxygen radical is greatest on complexes of the respiratory chain (Sarmah et al., 2019). Previous studies have shown that the activity of the different mitochondrial complexes is highly compromised following ischemia-reperfusion (Dave et al., 2008). Capros® treatment and prophylaxis was able to restore the compromised complex I and IV activity of ischemic rats. However, no significant improvement in the activity of complex II was observed with Capros®. An improvement in the mitochondrial respiratory capacity was also observed in stroke animals with treatment and prophylaxis with Capros®. The improvement was not significant under the conditions of the present Example. In an embodiment, the present invention is directed to a longer duration of prophylaxis with a P. emblica-containing composition such as Capros® to improve mitochondrial respiratory capacity.

Without being bound by theory, the above results show mechanism(s) by which Capros® may confer neuroprotection and/or attenuate brain injury post-ischemic stroke. Expression of trophic factors post stroke is decreased, as demonstrated by the reduction in the expression of SDF-1 and BDNF. Trophic factors play a crucial role in modulating neuronal functions, which are compromised post-stroke (Gutiérrez-Fernández et al., “Trophic factors and cell therapy to stimulate brain repair after ischaemic stroke” Journal of Cellular and Molecular Medicine 16:2280-2290 (2012)). Capros® was able to elevate SDF-1 and/or BDNF levels when given as prophylaxis and as treatment, as shown in FIG. 16. BDNF through the TrkB-PI3K pathway can activate several downstream mediators that protect neurons against the detrimental effects of an ischemic insult (Gutierrez-Fernandez et al., 2012). BDNF is said to regulate the expression of GAP-43, which is involved in regulating neuronal growth and axonal regeneration (Fournier et al., “Brain-derived neurotrophic factor modulates GAP-43 but not tα1 expression in injured retinal ganglion cells of adult rats.” Journal of Neuroscience Research 47:561-572 (1997)). Capros® increased the levels of GAP-43 in ischemic rats. Without being bound by theory, neuroprotection provided by Capros® according to the present invention may include facilitating neurogenesis.

It was also observed that Capros® increased the expression of VEGF, which is neuroprotective and pro-angiogenic (Greenberg and Jin, “Vascular endothelial growth factors (VEGFs) and stroke” Cellular and Molecular Life Sciences 70:1753-1761 (2013)). Although VEGF levels are upregulated post stroke, Capros® was able to upregulate the expression significantly as compared to Stroke rats. ROCK2 is an important protein which is involved in regulating cytoskeletal dynamics and other cellular functions, expression of which is upregulated following ischemia (Niego et al., “Selective inhibition of brain endothelial Rho-kinase-2 provides optimal protection of an in vitro blood-brain barrier from tissue-type plasminogen activator and plasmin” PLoS One, 12(5): e0177332. https://doi.org/10.1371/journal.pone.0177332 (2017)), Hyun Lee et al., “Selective ROCK 2 inhibition in focal cerebral ischemia” Annals of Clinical and Translational Neurology 1:2-14 (2014)). Capros® normalized the expression of ROCK2 in ischemic rats. In a study on myocardial ischemia-reperfusion injury, Capros® demonstrated cardio-protective effects by upregulating the PI3K/Akt/GSK3β pathway (Thirunavukkarasu et al., 2015). In the current study, Capros® also upregulated the pathway as demonstrated by the increase in the expression of PI3K and GSK3β.

Thus, the P. emblica-containing composition, Capros®, confers neuroprotection by a) increasing the expression of neurotrophic factors like SDF-1, BDNF and VEGF; b) regulating neuronal growth and axonal regeneration as demonstrated by the increased expression of GAP-43, and in an embodiment, facilitating neurogenesis; and c) upregulating the PI3K/Akt/GSK3β pathway. The neuroprotective effects of a P. emblica-containing composition of this invention are further confirmed by the improvement in motor-functional coordination, reduction in infarct size and improvement in oxidative stress outcomes.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A method of providing neuroprotection from stroke injury in the brain of a subject comprising the steps of:
a. providing a composition comprising a Phyllanthus emblica extract, and
b. administering an effective amount of the composition to the subject to act on the subject's brain and provide neuroprotection from injury from stroke in the brain.

2. The method of claim 1, wherein the composition comprises a standardized aqueous extract of Phyllanthus emblica.

3. The method of claim 2, wherein the composition is a dietary supplement.

4. The method of claim 3, wherein the composition is Capros.

5. The method of claim 1, wherein said administering step b is prior to a stroke.

6. The method of claim 1, wherein said administering step b is after a stroke.

7. The method of claim 6, wherein said administering step b occurs about 1 hour or less after a stroke.

8. A method of attenuating brain injury from stroke in a subject comprising the steps of:
a. providing a composition comprising a Phyllanthus emblica extract, and
b. administering an effective amount of the composition to the subject to act on the subject's brain and attenuate injury from stroke in the subject's brain.

9. The method of claim 8, wherein the composition comprises a standardized aqueous extract of Phyllanthus emblica.

10. The method of claim 9, wherein the composition is a dietary supplement.

11. The method of claim 10, wherein the composition is Capros.

12. The method of claim 8, wherein said administering step b is prior to a stroke.

13. The method of claim 8, wherein said administering step b is after a stroke.

14. The method of claim 13, wherein said administering step b occurs about 1 hour or less after a stroke.

15. A method of providing neuroprotection or attenuating injury from a cognition-related disease or disorder in the brain of a subject comprising the steps of:
a. providing a composition comprising a Phyllanthus emblica extract, and
b. administering an effective amount of the composition to the subject to act on the subject's brain and provide neuroprotection and/or attenuate injury from cognition-related disease or disorder in the brain.

16. The method of claim 15, wherein said disease or disorder is mild cognitive impairment or dementia.

17. The method of claim 15, wherein said disease or disorder is Huntington's disease, Alzheimer's disease, and/or vascular dementia.
